(19) United States
(12) Patent Application Publication
(10) Pub. No.: US 2020/0330711 A1
(43) Pub. Date: Oct. 22, 2020

(54) ATRAUMATIC NASOENDOTRACHEAL TUBE

(71) Applicant: Ryan Johansen, West Jordan, UT (US)

(72) Inventor: Ryan Johansen, West Jordan, UT (US)

(21) Appl. No.: 16/851,406

(22) Filed: Apr. 17, 2020

Related U.S. Application Data
(60) Provisional application No. 62/836,547, filed on Apr. 19, 2019.

(51) Int. Cl. A61M 16/04

(52) U.S. Cl. CPC A61M 16/0461; A61M 2210/0618; A61M 25/0068; A61M 2210/1028; A61M 2210/1032

(57) ABSTRACT

A nasoendotracheal tube including: a distal end with an atraumatic tapered profile, and a separation region located between the distal end and a proximal end of the nasoendotracheal tube, the separation region to separate under tension causing the distal end to separate from the proximal end.

### BACKGROUND

Airway access during oral surgery and other medical procedures of the mouth may be maintained using a nasotracheal catheter, a type of endotracheal tube. A nasotracheal catheter passes through the nose to the trachea, passing through the nasal cavity, pharynx, and larynx respectively. The nasotracheal catheter may be used to provide an unobstructed path for breathing, for example, when the mouth contains fluid such as saliva, saline, and/or blood. However, inserting a nasotracheal catheter without injury to the patient can be a challenge. It is not uncommon for insertion of a nasotracheal catheter to produce bleeding and other problems which make the associated medical activity in the mouth more challenging.

### BRIEF DESCRIPTION OF THE DRAWINGS

The accompanying drawings illustrate various examples of the principles described herein and are a part of the specification. The illustrated examples do not limit the scope of the claims.

FIG. 1A shows an example of a nasoendotracheal tube consistent with this specification.

FIG. 1B shows an example of a nasoendotracheal tube consistent with this specification.

FIGS. 2A-2D shows examples of separation zones for a nasoendotracheal tube consistent with this specification.

FIG. 3 shows an example of a nasoendotracheal tube consistent with this specification.

FIG. 4 shows a cross-section of a separation zone for a nasoendotracheal tube consistent with this specification.

FIG. 5 shows a flowchart for a method of using a nasoendotracheal tube consistent with this specification.

FIG. 6 shows an example of a separation zone for a nasoendotracheal tube consistent with this specification.

FIG. 7 shows the relevant anatomic regions.

FIG. 8 shows the relevant anatomic regions with the nasoendotracheal tube partially inserted.

FIG. 9 shows the relevant anatomic regions with the nasoendotracheal tube inserted to the point where the separation region may be separated, prior to advancing the nasoendotracheal tube into the trachea.

Throughout the drawings, identical reference numbers designate similar, but not necessarily identical, elements. The figures are not necessarily to scale, and the size of some parts may be exaggerated or minimized to more clearly illustrate the example shown. The drawings provide examples and/or implementations consistent with the description. However, the description is not limited to the examples and/or implementations shown in the drawings.

### DETAILED DESCRIPTION

A nasoendotracheal catheter is inserted through the nose and guided to the trachea via the larynx to provide airway support and/or access. One challenge is that the tip of the tube may cause injury during insertion. This may result in bleeding and other issues, generally minor, but which make the subsequent medical operations more challenging. For example, bleeding may make viewing an area being treated in the mouth more challenging to visualize. Similarly, blood may make surfaces harder to grasp or manipulate. Bleeding may also make it more challenging for the anesthesiologist to pass the end of the nasoendotracheal catheter through the vocal cords.

Nasoendotracheal intubation is used for various surgical procedures including, for example, those provided by ear nose and throat surgeons, craniofacial plastic surgeons, oral and maxillofacial surgeons, and dentistry. An anesthesiologist sedates the patient and places an endotracheal tube nasally to intubate the patient. This is done so that any oral surgery or throat surgery can be done without the endotracheal tube being in the way of the surgery and while maintaining an effective and patent airway during the procedure.

Most commonly the anesthesiologist will place a plain endotracheal tube thru a naris and into the nasal cavity down thru the nasopharynx and into the larynx placed between the vocal chords. This will maintain a stable airway during the procedure. Inserting a standard nasoendotracheal tube often traumatizes the tender tissues in the nasal cavity, nasopharynx, and posterior oropharynx. This results in a bloody field of view for an anesthesiologist during intubation and for the surgeon before the procedure begins. There is also additional postoperative pain and discomfort for the patient from the damage from the nasoendotracheal tube.

One approach used to reduce trauma to the surrounding tissue during intubation is to place a red rubber catheter over the tip of the nasoendotracheal tube. This facilitates passage of the nasoendotracheal tube thru the structures in an atraumatic way. However, this approach is cumbersome and time consuming, leading many anesthesiologists to place nasoendotracheal tubes without use of the red rubber catheter. The additional time to perform this procedure is considered undesirable as there is a consensus that reducing the intubation time is important to reduce risk to the patient.

Procedure for nasoendotracheal catheter placement: Anesthesiologist sedates the patient under general anesthesia. Anesthesiologist then inserts the blunt end of the ET tube into the nose and forces it thru the nasal cavity and nasopharynx. A laryngoscope is then used to find the end of the endotracheal tube and then proceeds to intubate the patient usually using McGill forceps. This is the quickest way to intubate the patient. However forcing the rigid tube past the delicate nasal tissues can cause great trauma to the mucosal membranes and adenoids and produce significant bleeding. This trauma and bleeding may slow down the intubation procedure by requiring suction to clear the field of view.

Procedure with Red-Rubber Catheter: Anesthesiologist sedates the patient under general anesthesia. The narrow tip of the red rubber catheter is place thru the nares back to the oropharynx. The anesthesiologist then uses a laryngoscope and McGill forceps to reach into the oral cavity and grab the end of the red rubber catheter. The anesthesiologist then pulls the end of the catheter out of the mouth until the other end of the catheter is at the nares where he or she places the end of the endotracheal tube inside the catheter and then advances the catheter and endotracheal tube together passing the nasal cavity and nasopharynx without traumatizing the mucosal membranes. Once the two together are advanced passed the narrow nasopharynx and/or delicate tissues the anesthesiologist then pulls on the tip of the red rubber catheter separating it from the endotracheal tube. The anesthesiologist can then proceed with the final intubation process with a clear field of view.

Procedure with the Atraumatic Nasoendotracheal Tube (ANET): Anesthesiologist sedates the patient under general anesthesia. Anesthesiologist then places the tapered end of the ANET tube into a naris and back thru the nasopharynx and into the oropharynx. The anesthesiologist may then use a laryngoscope and/or McGill forceps to grab the tapered end of the ANET tube. When the anesthesiologist pulls on the tapered end and in an opposite direction on the other end of the ANET tube, the tapered end separates from the nasoendotracheal tube. The anesthesiologist then proceeds to intubate the patient in a clean and clear field of view.

The Atraumatic Nasoendotracheal (ANET) tube reduces or eliminates trauma to the tissue during insertion. The ANET includes a proximal portion similar to a radiopaque endotracheal tube. However, a distal portion, including the tip, extends and tapers to a narrow end. The end of the endotracheal tube and tapered end are attached with a separation area. In an example, the separation area is a crimped area where the tube material is thin and perforated, allowing the anesthesiologist to separate the tapered end from the tube.

The inventor provides the following statement about the expected benefit of the proposed device. “Having 15 years of experience as a pediatric dentist, I have done thousands of procedures requiring general anesthesia. I have watched the anesthesiologist intubate the patient, with few of them using the red rubber catheter. As a result, most of the time, I began a procedure with the patient having a mouth full of blood from a traumatic nasotracheal intubation. With thousands of these dental procedures done daily, as well as Ear, Nose, and Throat (ENT) and Crainio-facial surgical procedures, the benefits of an integrated, guiding, separatable distal region on a nasoendotracheal tube could be wide spread.”

Among other examples, this specification describes a nasoendotracheal tube including: a distal end with an atraumatic profile, and a separation region located between the distal end and a proximal end of the nasoendotracheal tube, the separation region to separate under tension causing the distal end to separate from the proximal end.

Among other examples, this specification also describes a method of intubating a patient, including: grasping a distal region of a nasoendotracheal tube in a mouth of a patient; holding a proximal region of the nasoendotracheal tube; and applying tension between the proximal region and distal region of the nasoendotracheal tube to cause a body of the nasoendotracheal tube to separate.

This specification also describes a nasoendotracheal tube including: a distal end with a rounded profile, and a separation region located between the distal end and a proximal end of the nasoendotracheal tube, the separation region comprising perforations in a wall of the nasoendotracheal tube, wherein the separation region separates under tension causing the distal end to separate from the proximal end.

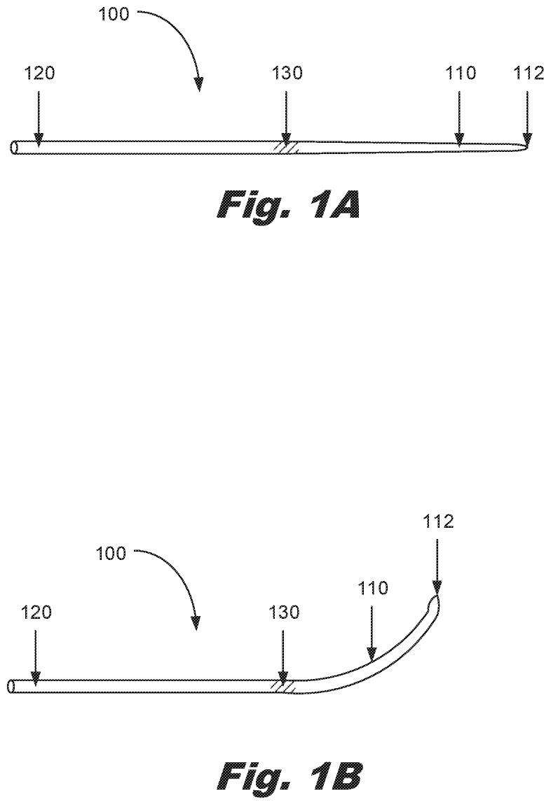

Turning now to the figures, FIG. 1A shows a nasoendotracheal tube (100) including: a distal end (110) with an atraumatic, tapered profile (112), and a separation region (130) located between the distal end (110) and a proximal end (120) of the nasoendotracheal tube (100), the separation region (130) to separate under tension causing the distal end (110) to separate from the proximal end (120).

The nasoendotracheal tube (100) is an atraumatic nasoendotracheal tube (100). When placed, the nasoendotracheal tube (100) provides a connection between the separation region (130) and the proximal end (120). The connection may be used to provide a patent airway into the trachea of the patient once the separation region is advanced into the trachea.

The nasoendotracheal tube (100) has a distal end (110). The distal end (110) is the end of the nasoendotracheal tube (100) which is inserted into a patient. The proximal end (120) is the end of the nasoendotracheal tube (100) which remains external to the patient.

The distal end (110) of the nasoendotracheal tube (100) has an atraumatic profile (112). The atraumatic profile (112) includes a shape to allow the distal end to be advanced through the nasal cavity and nasopharynx while minimizing damage to the tender mucosal membranes.

The distal end (110) of the nasoendotracheal tube (100) may include a softer material than a remainder of the nasoendotracheal tube (100). In another example, a stiffness of the distal end (110) of the nasoendotracheal tube (100) may be reduced compared with a stiffness of the proximal end (120) of the nasoendotracheal tube (100). This stiffness reduction may be due to differences in wall thickness, differences in material, post processing of the material, mechanical design features in the distal end (110) and/or proximal end (120) such as reinforcing ribs (e.g., on the proximal end (120)), or pleats to facilitate deflection (e.g., on the distal end (110)). Selective softening and/or stiffening may be added by secondary processes, for example, by crosslinking and/or polymer degradation using ultraviolet (UV) radiation. In some examples, the nasoendotracheal tube (100) is fabricated with different material properties on the different ends. For example, in a silicone nasoendotracheal tube (100), the proximal end may be heavily crosslinked to increase relative stiffness and enhance pushability. In some examples, the separation zone (130) may have a different color or translucence to make the separation zone (130) easier to see. For example, the separation zone may be colored white, red, black, or some other color compared with the rest of the nasoendotracheal tube (100). The coloration may be applied externally, internally, or mixed into the material. The coloration may be the result of a difference in crystallization in the separation zone. For example, the nasoendotracheal tube may be mostly amorphous but the separation zone (130) may be crystalized (and opaque) to make it easier to tear and/or separate.

The distal end (110) of the nasoendotracheal tube (100) may have a profile (112). The profile, including both the shape and stiffness of the tip of the distal end (110) may be selected to minimize injury while inserting the tube (100). The atraumatic tip (112) may have a bullet shape, be a blunt dissector, a rounded cone, and/or another similar shape to provide advancement without damage to the tissue. In an example, the atraumatic tip (112) is radially symmetrical. In another example, the atraumatic tip (112) is radially asymmetrical. For example, the atraumatic tip (112) may preferentially bend in one direction. This may be useful in managing the curve in the nasopharynx from the nasal cavity into the pharynx. The tube (100) may include a marker and/or other indicia to indicate a preferential insertion orientation. The tube (100) may include multiple markers and/or indicia, for example, one for a right naris insertion and a second for a left naris insertion. The markings may use the same marking as the separation zone (130) to reduce the number of body contacting materials.

The bias to the tube (100) may be sufficient to aid in tracking through the turn in the nasopharynx but still retain sufficient elastic recoil to have the tube (100) against a back wall of the pharynx in the oropharynx. In another example, the distal tip of the tube (100) may be biased to run along the proximal wall of the pharynx until it reaches the opening to the mouth (below the palatine tonsil, if present) and then track into the mouth. The separation region (130) may be neutrally biased so that once the tip is removed, the remainder of the endotracheal tube (100) advances down the oropharynx and laryngopharynx. This is another advantage of the described device over conventional tools, the ability to have one set of shape and/or biases for initial insertion through the nasopharynx and then a separate set of shape and/or biases to pass through the larynx once the distal end (110) of the nasoendotracheal tube (100) is removed. In contrast, a tube (100) without such a removable distal end (110) uses the same distal end to traverse the curve of the nasopharynx and the straight portion of the larynx, resulting in a compromise profile for one region and/or the other.

The proximal end (120) of the nasoendotracheal tube (100) is the end external to the patient and available for the medical professional to access. The proximal end (120) of the tube (100) includes an opening to a lumen connected to the separation region (130). When the distal end (110) and proximal end (120) are separated, the lumen connects to an opening at the new distal end (110) located at the separation section (130).

Prior to separation, the lumen may extend past the separation region (130) into the distal end (110) of the nasoendotracheal tube (100). In some examples, the lumen extends to the atraumatic profile (112) at the distal tip of the distal end (110) of the nasoendotracheal tube (100). In some examples, the distal end (110) and/or distal tip include an opening connecting to the lumen. This may be useful to determine help determine position of the distal tip. This may also provide an opening for airway access prior to separation of the distal end (110) from the proximal end (120).

The separation region (130) is located between the distal end (110) and proximal end (120) of the nasoendotracheal tube (100). The separation region is capable of being separated by the medical professional without injury to the patient. In an example, separation in induced by holding and/or pulling on the distal end (120) using forceps and holding and/or pulling on the proximal end (110). McGill forceps are well suited for holding and/or tensioning the distal end (110) of the nasoendotracheal tube (100) during separation. However, other forceps and/or other suitable tools may be used depending on the preferences of the practitioner and/or availability.

The separation region (130) may include a plurality of holes through a wall of the endotracheal tube (100). The plurality of holes may be perforations. The plurality of holes may be in a line. The plurality of holes may be in a staggered line. The separation region may be angled relative to a local, principle axis of the tube (100) such that, when separated, the tube (100) has an angled profile on its distal tip. The line of perforation may be at a non-right angle to the longitudinal axis of the tube (100). In an example, the non-right angle is between 30 and 60 degrees. The non-right angle may be approximately 45 degrees, for example, between 40 to 50 degrees. The use of a non-right angle may facilitate passage of the tube through the larynx.

The separation region (130) may have a locally reduced wall thickness of a wall of the endotracheal tube (100). The separation region (130) may have a maximum outer diameter smaller than the outer diameter of adjacent portions of the endotracheal tube (100). The separation region (130) may be designed to avoid catching or grabbing during insertion, for example, by using rounded edges.

FIG. 1B shows a nasoendotracheal tube (100) including: a distal end (110) with an atraumatic, tapered profile (112), and a separation region (130) located between the distal end (110) and a proximal end (120) of the nasoendotracheal tube (100), the separation region (130) to separate under tension causing the distal end (110) to separate from the proximal end (120).

In this example, the distal end (110) of the nasoendotracheal tube (100) is precurved to facilitate the turn at the end of the nasal cavity and into the oral cavity below. In contrast, the region just proximal of the separation region (130) may be straight and/or less biased/curved so as to facilitate passage into the trachea but not into the oral cavity. By using a removable lead, different curvatures can be prepared in the nasoendotracheal tube (100) for different portions of the track between the naris and the trachea past the vocal cords. Once the initial turn out of the nasal cavity is performed and the distal end (110) is tracked into the oral cavity, the curved/biased portion may be removed through the mouth by separating the separation region (130) and the second curvature used to navigate into the trachea.

In an example, the nasoendotracheal tube (100) is provided with a stylet in the lumen which facilitates insertion to the back of the nasal cavity. The stylet may then be stabilized and the nasoendotracheal tube (100) advanced off the stylet and recovering a bias or curvature to make the corner into the nasopharynx and/or oropharynx. The stylet may then be withdrawn. The stylet may extend to the distal tip (112). In other examples, the stylet may stop short of the distal tip (112). This may provide a softer tip when advancing the nasoendotracheal tube (100). In an example, the stylet is approximately 1 cm short of the distal tip (112) of the nasoendotracheal tube (100).

The use of a stylet also provides a first stiffness for the nasoendotracheal tube (100) when inserting the distal end (110) and a second stiffness after the stylet is removed and the nasoendotracheal tube (100) is advanced into the trachea. Allowing separate optimization of these stiffnesses may provide a design that is faster to insert than traditional designs which have a constant stiffness at the distal end of the nasoendotracheal tube (100).

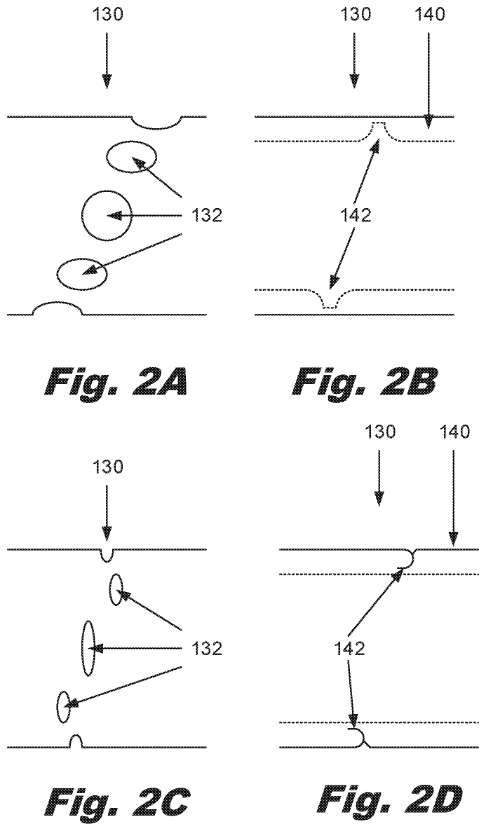

FIGS. 2A to 2D show examples separation regions (130) for nasoendotracheal tubes consistent with this specification. These represent some exemplary ways of implementing the separation region (130) but clearly other designs could readily be used to accomplish the separation.

FIG. 2A shows a separation region (130) having a series of perforations (132) with the perforations (132) being roughly circular. FIG. 2C shows a similar design but with non-circular perforations (132). The perforations (132) may be roughly linear as seen in FIG. 2A. The perforations (132) may be staggered and/or non-linear as seen in FIG. 2C. The perforations (132) may be at an angle, which is to say a non-right angle, to the longitudinal axis of the tube in the separation region (130). The spacing, size, and/or shape of the perforations (132) may be non-uniform, for example, the initial tear may have a first force threshold to initiate separation but the perforations (132) may then be closer so that the propagation force is lower. In another example, the final two or few perforations (132) may have a higher force than that propagation force to alert the medical professional that the distal end is now free from the endotracheal tube (100) and may be withdrawn through the mouth.

FIG. 2B shows a separation region (130) with an area of locally reduced wall thickness (142) of the wall (140) of the nasoendotracheal tube (100). In this example, the outer diameter is smooth. The profile may be inverted to dip in along the outer surface towards the inner wall of the tube (100), for example as seen in FIG. 2D. The wall profile may be combined with the perforations (132) and/or other mechanical elements.

FIG. 2D shows a separation region (130) with a profile such than when the separation region (130) separates, the resulting tip has a smooth profile in the tissue contacting areas. When the separation region (130) is stretched, the stress concentrates in the narrow region near the inner wall until it breaks and separates. Additional features may be added to localize crack initiation. For example, the inner surface may be scored or otherwise provided with a crack initiation site near the narrow region.

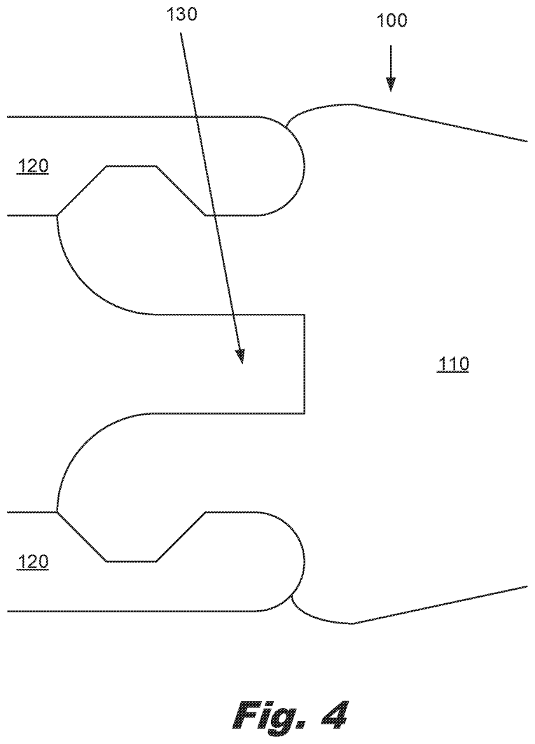

In some examples, the separation region (130) is formed as part of creating the tube of the nasoendotracheal tube (100). For example, the separation region may be formed by casting a silicone in a mold with a mandrel. The mandrel may have a release agent applied to facilitate separation from the resulting cast part. The separation region (130) may also be formed after formation of the tube. In this example, a thermoplastic tube is extruded. The tube is then shaped to a desired shape, included desired bends, etc. The separation region (130) may then be formed by inserting a mandrel into the inside of the tube in the separation region (130). A heated shaper may be applied the exterior of the tube in the separation region (130) to adjust the shape and form the features of the separation region, e.g. groves, perforations, etc. This approach tends to facilitate forming the features of the separation region on the exterior surface but can also be applied, with proper fixturing to the interior surface of the tube. Similar approaches may be used with the interlock designs such as those found in FIG. 4. In an example, a thermoplastic tube is flared over a shaped mandrel. The tube may then be then heated to conform the interior surface of tube to the mandrel and form the desired features. In some examples, an exterior crimp may be applied. The tube may be removed while hot. The tube may be allowed to cool and then the mandrel removed. In an example, the mandrel is an expandable mandrel which is unexpanded prior to removal to avoid stressing and or stretching the joint area.

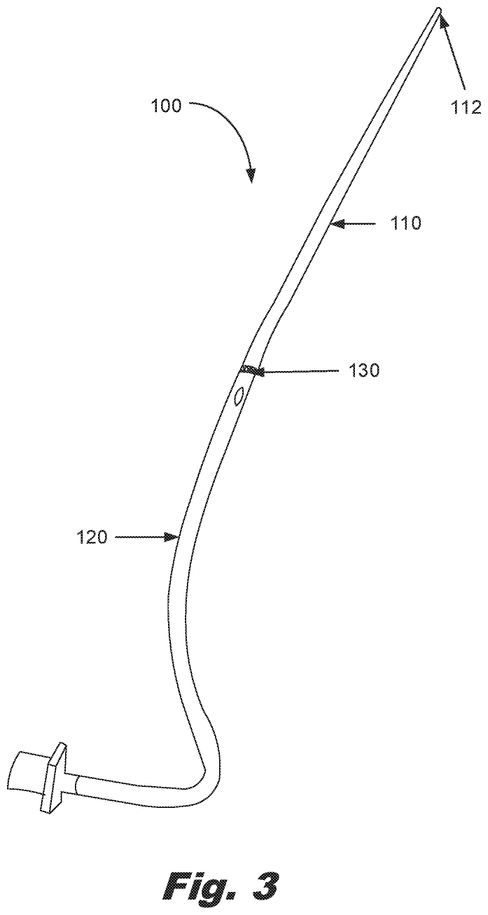

FIG. 3 shows a drawing of an implementation of a nasoendotracheal tube (100) consistent with this specification. The separation region (130) in this example includes a series of perforations at a non-right angle to the local longitude of the tube (100). The tube (100) includes an opening allowing verification of ventilation prior to separation of the distal region (110) from the proximal region (120). This particular figure also shows a standard connection on the proximal end of the nasoendotracheal tube (100). The standard connection may be provided with the tube (100) the standard connection may be added by a medical professional, e.g., anesthesiologist. The standard connection may be removable. The standard connection may be integrated with the rest of the nasoendotracheal tube (100), for example, with adhesive, friction fit, mechanical interlock, etc.

FIG. 3 includes an opening (332) proximal of the separation region (130). The opening (332) may provide an alternate airflow path in the event of obstruction of the tip of nasoendotracheal tube (100) after separation of the separation region (130).

FIG. 4 shows a cross-section view of a separation region (130) of a nasoendotracheal tube (100) consistent with this specification. In this example, the distal region (110) and proximal region (120) are physically separate pieces that are latched together during insertion. An approach like this may allow better control of the resulting shape of the tip which remains after separation of the distal region (110).

In some examples, the two pieces are connected with an adhesive and/or another material to form an integral tube during insertion. The adhesive may be, for example, a low durometer silicone. In other examples, the connection is supported by a mechanical interlock and/or friction. In some examples, the distal region (110) has a largest diameter smaller than the typical diameter of the proximal region (120). The separation region (130) may similarly have the same and/or a smaller largest diameter compared to the distal region (110) and/or proximal region (120). This may reduce the likelihood that the separation region (130) will catch on an anatomic feature during insertion.

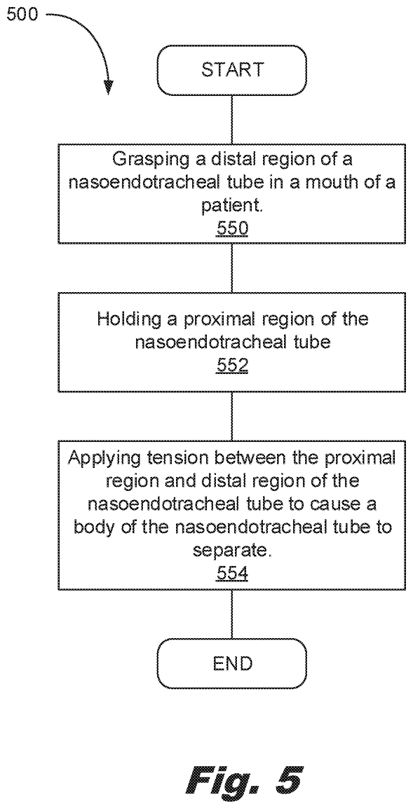

FIG. 5 shows a flowchart for a method (500) of using a nasoendotracheal tube (100) consistent with this specification. The method (500) includes: grasping (550) a distal region (110) of a nasoendotracheal tube (100) in a mouth of a patient; holding (552) a proximal region (120) of the nasoendotracheal tube (100); and applying tension (554) between the proximal region (120) and distal (110) regional of the nasoendotracheal tube (100) to cause a body of the nasoendotracheal tube (110) to separate.

The method (500) is a method of using a nasoendotracheal tube (100). The distal region (110) of the endotracheal tube is introduced through a naris and nasal cavity into the pharynx. The distal region (110) is then grasped through the mouth. The proximal region (120) is also stabilized and/or held. Tension is applied between the proximal (120) and distal (110) regions until the nasoendotracheal tube (100) separates at a separation region (130). The distal region (110) is then removed through the mouth. The proximal region (120) is advanced into the larynx to provide access to the trachea, for example, during an oral surgery.

The method (500) includes grasping (550) a distal region (110) of a nasoendotracheal tube (100) in a mouth of a patient. The mouth as used herein includes the oropharynx. Grasping the distal region (110) of the nasoendotracheal tube (100) may include grasping the distal region (110) with forceps. The forceps may be McGill forceps. In some examples, the distal region (110) is grasped near the tip of the distal region (110).

The method (500) includes holding (552) a proximal region (120) of the nasoendotracheal tube (100). The proximal region may be held by hand. The proximal region may be held by a tool. Holding both ends of the nasoendotracheal tube (100) allows the tension to be applied the body of the tube (100).

The method (500) includes applying tension (554) between the proximal region (120) and distal (110) regional of the nasoendotracheal tube (100) to cause a body of the nasoendotracheal tube (100) to separate. The separation may be result of breaking the body of the nasoendotracheal tube (100). The separation may be the result of unlatching and/or similarly disengaging the distal region (110) and proximal region (120). In an example, torsion is also applied to facilitate the separation.

The separation of the distal region (110) and proximal region (120) may be the localized at a separation region (130) connecting the distal region (110) and proximal region (120). The separation region (130) may include features to facilitate and/or localize the separation. For example, the separation region (130) may have a locally reduced wall thickness. The separation region (130) may have latches or similar features to hold the distal region (110) and proximal region (120) together where the latches or similar features disengage at a tension below a tension to break the nasoendotracheal tube. The separation region (130) may have holes, perforations, scoring, notches, and/or other features to initiate local breaking and/or separation. The feature(s) may function as stress concentrators. The features may reduce the local load bearing strength of the wall of the nasoendotracheal tube (100). The features may be designed to leave a smooth distal tip of the proximal region (120) after separation. The distal tip of the proximal region (120) may be form a non-right angle relative to the longitudinal axis of the tube (100). In one example, the non-right angle is between 30 and 60 degrees relative to the longitudinal axis of the tube (100).

The method may further include removing the distal region of the nasoendotracheal tube from the mouth of the patient using the forceps. This allows continuous holding of the distal region (110) of the nasoendotracheal tube (100) limiting the risk of the distal region (110) going to an undesired location. The method (500) may include having the forceps continuously hold the distal region of the nasoendotracheal tube between grasping the distal end, through breaking the body of the nasoendotracheal tube, and removal of the distal region (110) of the nasoendotracheal tube (100) from the mouth of the patient. While this is optional, for example, the distal region (110) could be dropped in the mouth and then picked up and removed; holding the distal region (110) continuously reduces the risk that the distal portion (110) goes somewhere undesirable.

In some examples, the body of the nasoendotracheal tube includes multiple holes in a wall of the body of the endotracheal tube and the method further includes verifying a hole of the multiple holes are can provide air to the patient prior to breaking the body of the nasoendotracheal tube. The ability to verify that the endotracheal tube is in a proper position by ventilating through a hole prior to separating the endotracheal tube provides an additional level of safety. If a proper position for ventilation cannot be found, the nasoendotracheal tube can be withdrawn through the nasal cavity and naris instead of separating.

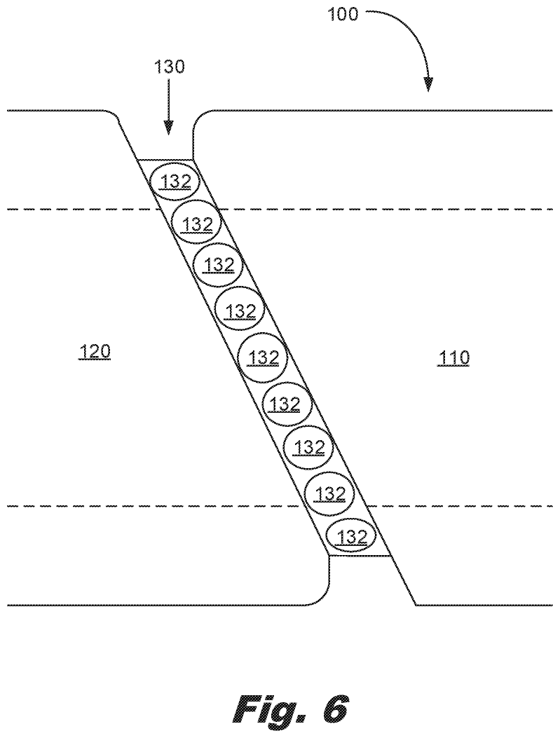

FIG. 6 shows an example of a separation region (130) for nasoendotracheal tubes (100) consistent with this specification. The figure shows the nasoendotracheal tube (100) having a distal region (110) and a proximal region (120) connected by a separation region (130). The separation region has a reduced diameter compared with the adjacent portions of the distal region (110) and proximal region (120). In this example, the diameter of the lumen in the tube is uniform through the separation region. The separation region (130) contains a number of perforations (132) which facilitate separation of the distal region (110) from the proximal region (120) when the distal end of the distal region (110) has passed through the naris and nasal cavity of the patient to the oropharynx and/or mouth.

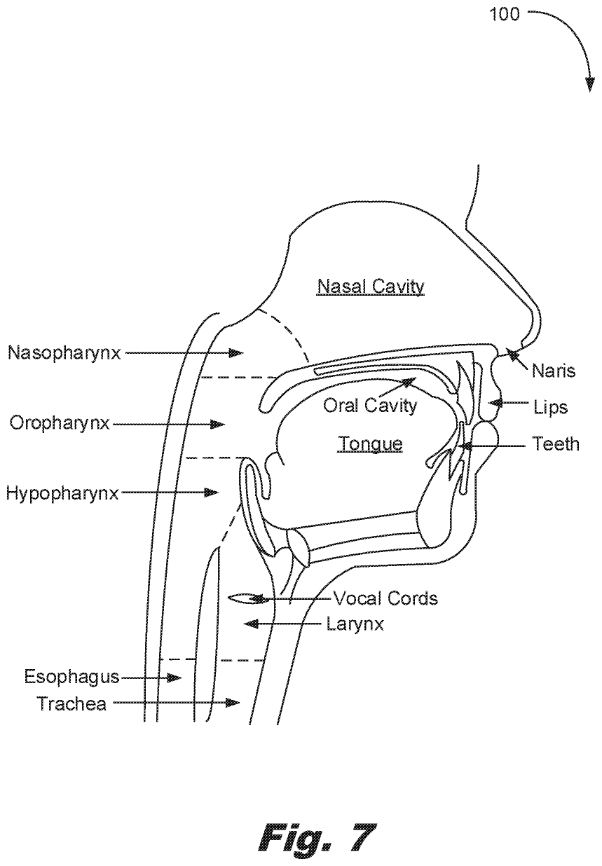

FIG. 7 shows the relevant anatomic regions.

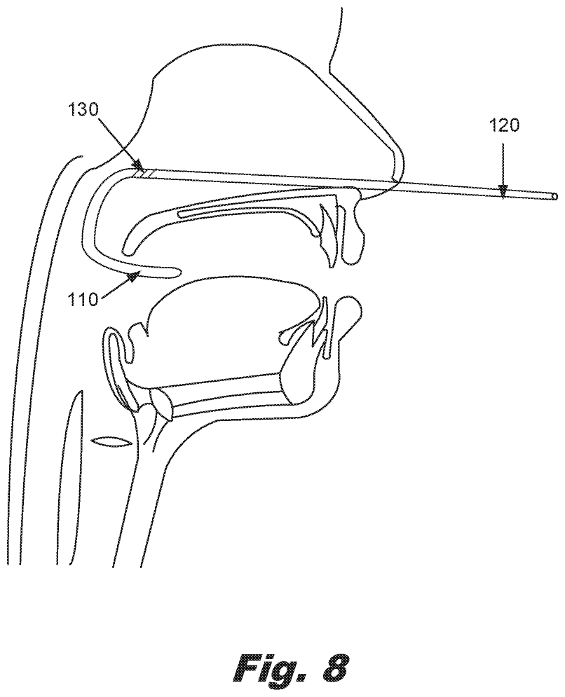

FIG. 8 shows the relevant anatomic regions with the nasoendotracheal tube (100) partially inserted.

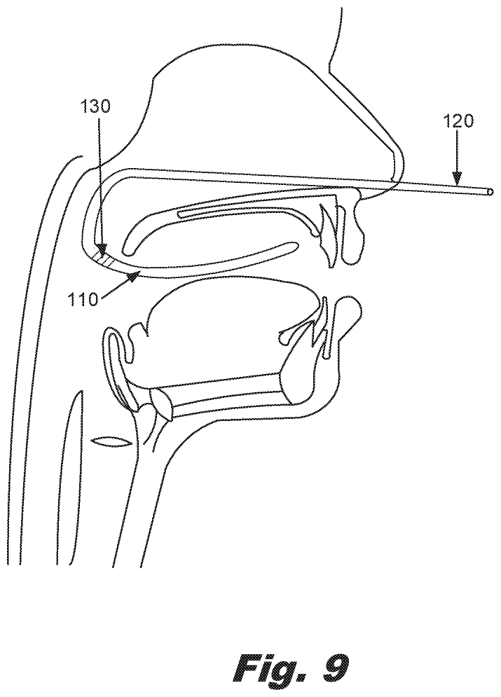

FIG. 9 shows the relevant anatomic regions with the nasoendotracheal tube (100) inserted to the point where the separation region (130) may be separated, prior to advancing the nasoendotracheal tube (100) into the trachea.

It will be appreciated that, within the principles described by this specification, a vast number of variations exist. It should also be appreciated that the examples described are only examples, and are not intended to limit the scope, applicability, or construction of the claims in any way.

What is claimed is:

1. A nasoendotracheal tube comprising:
a distal end with an atraumatic tapered profile, and
a separation region located between the distal end and a proximal end of the nasoendotracheal tube, the separation region to separate under tension causing the distal end to separate from the proximal end.

2. The nasoendotracheal tube of claim 1, wherein the separation region comprises a plurality of holes through a wall of the endotracheal tube.

3. The nasoendotracheal tube of claim 1, wherein the separation region comprises a locally reduced wall thickness of a wall of the endotracheal tube.

4. The nasoendotracheal tube of claim 1, wherein the separation region is angled relative to a principle axis of the tube such that, when separated, the tube has an angled profile.

5. The nasoendotracheal tube of claim 4, wherein the separation region comprises a series of perforations.

6. The nasoendotracheal tube of claim 1, wherein the atraumatic tip has a bullet shape.

7. The nasoendotracheal tube of claim 1, wherein the atraumatic tip is a rounded cone.

8. The nasoendotracheal tube of claim 1, wherein the distal end is curved.

9. A method of intubating a patient, comprising:
grasping a distal region of a nasoendotracheal tube in a mouth of a patient;
holding a proximal region of the nasoendotracheal tube; and
applying tension between the proximal region and distal regional of the nasoendotracheal tube to cause a body of the nasoendotracheal tube to separate.

10. The method of claim 9, wherein grasping the distal region of the nasoendotracheal tube comprises grasping the distal end with forceps.

11. The method of claim 10, further comprising removing the distal region of the nasoendotracheal tube from the mouth of the patient using the forceps.

12. The method of claim 11, wherein the forceps continuously hold the distal region of the nasoendotracheal tube between grasping the distal end, through breaking the body of the nasoendotracheal tube, and removal of the distal region of the nasoendotracheal tube from the mouth of the patient.

13. The method of claim 9, wherein the body of the nasoendotracheal tube comprises multiple holes in a wall of the body of the endotracheal tube.

14. A nasoendotracheal tube comprising:
a distal end with a rounded profile, and
a separation region located between the distal end and a proximal end of the nasoendotracheal tube, the separation region comprising perforations in a wall of the nasoendotracheal tube, wherein the separation region separates under tension causing the distal end to separate from the proximal end.

15. The nasoendotracheal tube of claim 14, wherein a wall thickness in the separation region is narrower than a wall thickness in an adjacent region.
